(19) United States
(12) Patent Application Publication
(10) Pub. No.: US 2021/0352887 A1
(43) Pub. Date: Nov. 18, 2021

(54) OXYGENATION MEDIA FOR EX-VIVO PRESERVATION OF ORGANS AND TISSUES

(71) Applicant: VirTech Bio, Inc., Natick, MA (US)

(72) Inventor: William Richard Light, Natick, MA (US)

(73) Assignee: VirTech Bio, Inc., Natick, MA (US)

(21) Appl. No.: 17/284,668

(22) PCT Filed: Oct. 11, 2019
(86) PCT No.: PCT/US2019/055797

§ 371 Date: Apr. 12, 2021

Related U.S. Application Data
(60) Provisional application No. 62/745,020, filed on Oct. 12, 2018.

(51) Int. Cl. A01N 1/02

(52) U.S. Cl. CPC A01N 1/021

(57) ABSTRACT

Oxygenation constituents and finished oxygenation media, as well as methods of making oxygenation constituents and finished oxygenation media are provided. The oxygenation constituents comprise a hemoglobin preparation, and a red blood cell preparation. The oxygenation constituents comprise from about 10% to about 99% by weight hemoglobin of the hemoglobin preparation and the balance by weight hemoglobin of the oxygenation constituents comprise a red blood cell preparation. The finished oxygenation media comprise the oxygenation constituents and one or more other ingredients such as a diluent, or excipient. The finished oxygenation media can be used to ex-vivo preserve organs or tissue.

### FIELD OF THE DISCLOSURE

The present disclosure generally relates to oxygenation media, and in particular, to oxygenation media and methods of making and using oxygenation media for preserving tissues and organs ex-vivo.

### BACKGROUND

The following paragraphs are provided by way of background to the present disclosure. They are not however an admission that anything discussed therein is prior art or part of the knowledge of persons skilled in the art.

It is frequently desirable in medical practice to preserve organs or tissue outside of a living subject. Thus, for example, vital organs, such as kidney, lung or heart, may be removed from a transplant donor and temporarily maintained separated from a living subject, a condition commonly referred to as “ex-vivo”, prior to clinical transplantation to a transplant recipient. It is well known that in order to maintain ex-vivo viability, it is of importance that organs or tissues remain sufficiently oxygenated. In the absence of adequate oxygen supply cell metabolism ceases, leading to cell damage, and eventually cell and tissue death.

Accordingly, several techniques for oxygen supply compatible with ex-vivo preservation of organs and tissue have been developed. Thus, for example, an organ or tissue can be preserved in a holding container comprising a liquid medium. Oxygenation of the medium may be achieved by including a circulation system, i.e. a pump, configured to more or less continuously supply oxygen to the liquid medium. Systems designed to achieve organ or tissue preservation in this manner are known as machine perfusion systems.

Various oxygen carrying media have evolved with efforts generally being directed at prolonging the time during which organs and tissues can be safely ex-vivo preserved. Thus, for example blood, and its oxygen carrying constituents red blood cells and hemoglobin, as well as chemically modified hemoglobin derivatives, also known as hemoglobin based oxygen carriers or HBOCs, have been used to preserve organs and tissue ex-vivo. However there are significant drawbacks associated with the use of blood-based oxygen carriers known to the prior art. For example, the use of whole blood requires the operation at, or at near physiological temperatures, i.e. 37° C., and depends on the availability of sufficient quantities of donated blood. Furthermore red blood cell viability, red blood cell lysis, coagulation, and the presence of biological contaminants (notably, contaminants transmitting blood borne diseases, e.g. hepatitis B and human immunodeficiency virus (HIV)) all restrict the utility of known blood based oxygen carriers, and, in particular, the time during which organs and tissue can be preserved ex-vivo when these media are used.

A significant challenge associated with the use of hemoglobin and HBOCs as oxygen carriers arises from observations that certain quantities of hemoglobin, when isolated from red blood cells, are converted to a variant hemoglobin known as methemoglobin. In methemoglobin the iron within the protein's heme group, which serves as the molecular binding site for oxygen, is present in the ferric (Fe³⁺) state, and not in the ferrous (Fe²⁺), state. Methemoglobin is unable to bind oxygen and is therefore unable to serve as an oxygen carrier. Furthermore, methemoglobin is known to be prone to denaturation with the heme group dissociating from the protein, and unfolding of the protein. The unfolded protein can negatively affect hemoglobin preparations, including by the formation of precipitates.

Another approach to providing an oxygen carrying medium known to the art involves the use of perflurocarbon-based synthetic molecules capable of solubilizing oxygen. Synthetic carbon-fluorine molecules are chemically inert and straightforward to manufacture, however their oxygen solubility is low relative to hemoglobin, and perflurocarbon preparations are virtually immiscible with water and therefore require emulsification which render perflurocarbon preparations unstable and difficult to store.

Alternate efforts have focused on preserving tissue and organs at non-physiological temperatures, for example at less than 8° C., to halt cellular metabolism of the organ or tissues, however these techniques result in cell damage and prolonged presence at low temperatures results in organ or tissue death. Furthermore, at these temperatures, data relating to the tissue or organs obtained for diagnostic or research and development purposes may not be physiologically relevant.

Yet other techniques to preserve organs and tissues ex-vivo involve the exposure of organs or tissue to hyperbaric oxygen, or to high partial oxygen pressures. The use of these techniques however does generally not solve challenges when larger organs or tissues are used, and in particular cells located to the interior of the organs or tissue are subject to oxygen depletion, or the challenges associated with preservation at non-physiological temperatures.

Thus, it will be clear that although techniques for preserving organs and tissue ex-vivo involving the use of oxygen carrying media are known to the art, many drawbacks associated with known oxygen carrying media remain. There is therefore a need in the art for improved oxygen carrying media and methods of making oxygen carrying media, and in particular, there is a need for improved oxygen carrying media for the ex-vivo preservation of tissues and organs.

### SUMMARY

The following paragraphs are intended to introduce the reader to the more detailed description that follows and not to define or limit the claimed subject matter of the present disclosure.

In one broad aspect, the present disclosure relates to oxygenation media for the ex-vivo preservation of organs and tissue. Accordingly, in one aspect, in accordance with the teachings herein, the present disclosure provides, in at least one embodiment, an oxygenation constituent for formulating a finished oxygenation medium, the oxygenation constituent comprising a mixture of a first and second preparation wherein:
(i) the first preparation is a hemoglobin preparation comprising non-modified hemoglobin, HBOC, or a mixture thereof in an amount of from about 1% to about 40% by weight hemoglobin, the balance of the hemoglobin preparation comprising an aqueous hemoglobin diluent; and
(ii) the second preparation is a red blood cell preparation comprising hemoglobin in an amount of from about 1% to about 40% by weight hemoglobin, and the balance of the red blood cell preparation comprising an aqueous red blood cell diluent; and wherein
   the mixture is formed such that the oxygenation constituent comprises an amount of from about 10% to about 99% by weight hemoglobin of the hemoglobin preparation, and an amount equal to the balance by weight hemoglobin of the red blood cell preparation.

In at least one embodiment, the oxygenation constituent can comprise from about 20% to about 90% by weight hemoglobin of the hemoglobin preparation.

In at least one embodiment, the oxygenation constituent can comprise from about 30% to about 80% by weight hemoglobin of the hemoglobin preparation.

In at least one embodiment, the oxygenation constituent can comprise from about 40% to about 70% by weight hemoglobin of the hemoglobin preparation.

In at least one embodiment, the oxygenation constituent can comprise from about 50% to about 60% by weight hemoglobin of the hemoglobin preparation.

In at least one embodiment, the hemoglobin preparation can comprise non-modified hemoglobin, HBOC, or a mixture thereof in an amount of from about 5% to about 35% by weight hemoglobin.

In at least one embodiment, the hemoglobin preparation can comprise non-modified hemoglobin, HBOC, or a mixture thereof in an amount of from about 10% to about 30% by weight hemoglobin.

In at least one embodiment, the hemoglobin preparation can comprise non-modified hemoglobin, HBOC, or a mixture thereof in an amount of from about 15% to about 25% by weight hemoglobin.

In at least one embodiment, the red blood cell preparation can comprise hemoglobin in an amount of from about 5% to about 35% by weight hemoglobin.

In at least one embodiment, the red blood cell preparation can comprise hemoglobin in an amount of from about 10% to about 30% by weight hemoglobin.

In at least one embodiment, the red blood cell preparation can comprise hemoglobin in an amount of from about 15% to about 25% by weight hemoglobin.

In at least one embodiment, the oxygenation constituent can comprise:
(i) a hemoglobin preparation comprising non-modified hemoglobin, HBOC or a mixture thereof in an amount of from about 5% to about 15% by weight hemoglobin, the balance of the hemoglobin preparation comprising an aqueous hemoglobin diluent; and
(ii) a red blood cell preparation comprising hemoglobin in an amount of from about 10% to about 30% by weight hemoglobin, and the balance of the red blood cell preparation comprising an aqueous red blood cell diluent; and
wherein
   the mixture is formed such that the oxygenation constituent comprises an amount from about 40% to about 60% by weight hemoglobin of the hemoglobin preparation, and an amount equal to the balance by weight hemoglobin of the red blood cell preparation.

In at least one embodiment, the oxygenation constituent can comprise:
(i) a hemoglobin preparation comprising non-modified hemoglobin, HBOC or a mixture thereof in an amount of about 10% by weight hemoglobin, the balance of the hemoglobin preparation comprising an aqueous hemoglobin diluent; and
(ii) a red blood cell preparation comprising hemoglobin in an amount of about 20% by weight hemoglobin, and the balance of the red blood cell preparation comprising an aqueous red blood cell diluent; and
wherein
   the mixture is formed such that the oxygenation constituent comprises an amount of about 50% by weight hemoglobin of the hemoglobin preparation, and an amount equal the balance by weight hemoglobin of the red blood cell preparation.

In at least one embodiment, the hemoglobin preparation can comprise non-modified hemoglobin and is substantially free of HBOC.

In at least one embodiment, the hemoglobin preparation can comprise HBOC, and is substantially free of non-modified hemoglobin.

In at least one embodiment, the hemoglobin preparation can comprise a mixture of hemoglobin and HBOC.

In at least one embodiment, the aqueous hemoglobin diluent can be lactated Ringer's solution, or a modified form thereof.

In at least one embodiment, the aqueous red blood cell diluent can be lactated Ringer's solution, or a modified form thereof.

In at least one further aspect, the present disclosure provides in at least one embodiment, a finished oxygenation medium comprising:
(a) from about 1% by weight hemoglobin to about 39% by weight hemoglobin of an oxygenation constituent according to the present disclosure; and
(b) the balance of the medium comprising at least one other formulary ingredient selected from a diluent or an excipient, and optionally other ingredients suitable for formulating a finished oxygenation medium.

In at least one embodiment, the finished oxygenation medium can comprise:
(a) from about 1% by weight hemoglobin to about 39% by weight hemoglobin of an oxygenation constituent, the oxygenation constituent comprising a mixture of a first and second preparation wherein:
   (i) the first preparation is a hemoglobin preparation comprising non-modified hemoglobin, HBOC, or a mixture thereof in an amount of from about 1% to about 40% by weight hemoglobin, the balance of the hemoglobin preparation comprising an aqueous hemoglobin diluent; and
   (ii) the second preparation is a red blood cell preparation comprising hemoglobin in an amount of from about 1% to about 40% by weight hemoglobin, and the balance of the red blood cell preparation comprising an aqueous red blood cell diluent; and
   wherein
      the mixture is formed such that the oxygenation constituent comprises an amount of from about 10% to about 99% by weight hemoglobin of the hemoglobin preparation, and an amount equal to the balance by weight hemoglobin of the red blood cell preparation; and
(b) the balance of the medium comprising at least one other formulary ingredient selected from a diluent or an excipient, and optionally other ingredients suitable for formulating a finished oxygenation medium.

In at least one embodiment, the finished oxygenation medium can comprise from about 5% by weight hemoglobin to about 35% by weight hemoglobin of an oxygenation constituent.

In at least one embodiment, the finished oxygenation medium can comprise from about 10% by weight hemoglobin to about 30% by weight hemoglobin of an oxygenation constituent.

In at least one embodiment, the finished oxygenation medium can comprise from about 15% by weight hemoglobin to about 25% by weight hemoglobin of an oxygenation constituent, the oxygenation constituent comprising:
(i) a hemoglobin preparation comprising non-modified hemoglobin, HBOC or a mixture thereof in an amount of from about 5% to about 15% by weight hemoglobin, the balance of the hemoglobin preparation comprising an aqueous hemoglobin diluent; and
(ii) a red blood cell preparation comprising hemoglobin in an amount of from about 10% to about 30% by weight hemoglobin, and the balance of the red blood cell preparation comprising an aqueous red blood cell diluent; and wherein
   the mixture is formed such that the oxygenation constituent comprises an amount from about 40% to about 60% by weight hemoglobin of the hemoglobin preparation, and an amount equal to the balance by weight hemoglobin of the red blood cell preparation.

In at least one embodiment, the finished oxygenation medium can comprise about 20% by weight hemoglobin of an oxygenation constituent, the oxygenation constituent comprising:
(i) a hemoglobin preparation comprising non-modified hemoglobin, HBOC or a mixture thereof in an amount of about 10% by weight hemoglobin, the balance of the hemoglobin preparation comprising an aqueous hemoglobin diluent; and
(ii) a red blood cell preparation comprising hemoglobin in an amount of about 20% by weight hemoglobin, and the balance of the red blood cell preparation comprising an aqueous red blood cell diluent; and
wherein
   the mixture is formed such that the oxygenation constituent comprises an amount of about 50% by weight hemoglobin of the hemoglobin preparation, and an amount equal the balance by weight hemoglobin of the red blood cell preparation.

In at least one further aspect, the present disclosure further provides, in at least one embodiment, a method of preparing a finished oxygenation medium for ex-vivo use, the method comprising:
(i) providing a hemoglobin preparation comprising non-modified hemoglobin, HBOC, or a mixture thereof in an amount of from about 1% to about 40% by weight hemoglobin, the balance of the hemoglobin preparation comprising an aqueous hemoglobin diluent;
(ii) providing a red blood cell preparation comprising hemoglobin in an amount of from about 1% to about 40% by weight hemoglobin, and the balance of the red blood cell preparation comprising an aqueous red blood cell diluent;
(iii) mixing the hemoglobin preparation and the red blood cell preparation to form an oxygenation constituent such that the oxygenation constituent comprises an amount of from about 10% to about 99% by weight hemoglobin of the hemoglobin preparation, and an amount equal to the balance by weight hemoglobin of the red blood cell preparation; and
(iv) formulating the oxygenation constituent with at least one formulary ingredient selected from an excipient, and a diluent, and optionally other ingredients suitable for formulating a finished oxygenation medium.

In at least one embodiment, the finished oxygenation medium can comprise at least about 1% by weight of hemoglobin of the oxygenation constituent.

In at least one embodiment, the finished oxygenation medium can comprise from about 1% to about 39% by weight of hemoglobin of the oxygenation constituent.

In at least one embodiment, an amount of the hemoglobin preparation can be mixed such that the oxygenation constituent comprises an amount of from about 20% to about 90% by weight hemoglobin of the hemoglobin preparation.

In at least one embodiment, an amount of the hemoglobin preparation can be mixed such that the oxygenation constituent comprises from about 30% to about 80% by weight hemoglobin of the hemoglobin preparation.

In at least one embodiment, an amount of the hemoglobin preparation can be mixed such that the oxygenation constituent comprises from about 40% to about 70% by weight hemoglobin of the hemoglobin preparation.

In at least one embodiment, an amount of the hemoglobin preparation can be mixed such that the oxygenation constituent comprises from about 50% to about 60% by weight hemoglobin of the hemoglobin preparation.

In at least one embodiment, the hemoglobin preparation can comprise non-modified hemoglobin, HBOC, or a mixture thereof in an amount of from about 5% to about 35% by weight hemoglobin.

In at least one embodiment, the hemoglobin preparation can comprise non-modified hemoglobin, HBOC, or a mixture thereof in an amount of from about 10% to about 30% by weight hemoglobin.

In at least one embodiment, the hemoglobin preparation can comprise non-modified hemoglobin, HBOC, or a mixture thereof in an amount of from about 15% to about 25% by weight hemoglobin.

In at least one embodiment, the red blood cell preparation can comprise hemoglobin in an amount of from about 5% to about 35% by weight hemoglobin.

In at least one embodiment, the red blood cell preparation can comprise hemoglobin in an amount of from about 10% to about 30% by weight hemoglobin.

In at least one embodiment, the red blood cell preparation can comprise hemoglobin in an amount of from about 15% to about 25% by weight hemoglobin.

In at least one embodiment, the oxygenation constituent can comprise:
(i) a hemoglobin preparation comprising non-modified hemoglobin, HBOC or a mixture thereof in an amount of from about 5% to about 15% by weight hemoglobin, the balance of the hemoglobin preparation comprising an aqueous hemoglobin diluent; and
(ii) a red blood cell preparation comprising hemoglobin in an amount of from about 10% to about 30% by weight hemoglobin, and the balance of the red blood cell preparation comprising an aqueous red blood cell diluent; and
wherein
   the mixture is formed such that the oxygenation constituent comprises an amount from about 40% to about 60% by weight hemoglobin of the hemoglobin preparation, and an amount equal to the balance by weight hemoglobin of the red blood cell preparation; and
(iii) the finished oxygenation medium can comprise from about 15% by weight hemoglobin to about 25% by weight hemoglobin of the oxygenation constituent.

In at least one further aspect, the present disclosure further provides, in at least one embodiment, a use of an oxygenation constituent to formulate a finished oxygenation medium, the oxygenation constituent comprising a mixture of a first and second preparation wherein:
(i) the first preparation is a hemoglobin preparation comprising non-modified hemoglobin, HBOC, or a mixture thereof in an amount of from about 1% to about 40% by weight hemoglobin, the balance of the hemoglobin preparation comprising an aqueous hemoglobin diluent; and
(ii) the second preparation is a red blood cell preparation comprising hemoglobin in an amount of from about 1% to about 40% by weight hemoglobin, and the balance of the red blood cell preparation comprising an aqueous red blood cell diluent; and
wherein
   the mixture is formed such that the oxygenation constituent comprises an amount of from about 10% to about 99% by weight hemoglobin of the hemoglobin preparation, and an amount equal to the balance by weight hemoglobin of the red blood cell preparation.

In at least one further aspect, the present disclosure further provides, in at least one embodiment, a use of a finished oxygenation medium for the ex-vivo preservation of a tissue or an organ, the finished oxygenation medium comprising:
(a) from about 1% by weight hemoglobin to about 39% by weight hemoglobin of an oxygenation constituent, the oxygenation constituent comprising a mixture of a first and second preparation
wherein:
   (i) the first preparation is a hemoglobin preparation comprising non-modified hemoglobin, HBOC, or a mixture thereof in an amount of from about 1% to about 40% by weight hemoglobin, the balance of the hemoglobin preparation comprising an aqueous hemoglobin diluent; and
   (ii) the second preparation is a red blood cell preparation comprising hemoglobin in an amount of from about 1% to about 40% by weight hemoglobin, and the balance of the red blood cell preparation comprising an aqueous red blood cell diluent; and
wherein
   the mixture is formed such that the oxygenation constituent comprises an amount of from about 10% to about 99% by weight hemoglobin of the hemoglobin preparation, and an amount equal to the balance by weight hemoglobin of the red blood cell preparation; and
(b) the balance of the medium comprising at least one other formulary ingredient selected from a diluent or an excipient, and optionally other ingredients suitable for formulating a finished oxygenation medium.

In at least one embodiment, the ex-vivo preservation can be static ex-vivo preservation.

In at least one embodiment, the ex-vivo preservation can be dynamic ex-vivo preservation.

In at least one embodiment, the ex-vivo preservation can be dynamic ex-vivo preservation involving the use of a machine perfusion system configured to comprise a filtering device to filter the finished oxygenation medium.

In at least one embodiment, the ex-vivo preservation can be dynamic ex-vivo preservation involving the use of a machine perfusion system configured to comprise a continuous or semi continuous supply assembly for the finished oxygenation medium, or an excipient or diluent, and a filtering device to continuously filter the finished oxygenation medium.

In at least one embodiment, the filtering device can be a tangential flow based filtering device.

In at least one embodiment, the organ can be an organ for transplantation to a transplant recipient.

In at least one embodiment, the organ can be a lung, a heart, a liver or a kidney.

In at least one embodiment, the organ can be a human organ.

In at least one embodiment, the tissue can be for grafting to a graft recipient.

In at least one embodiment, the tissue can be for tissue engineering.

In accordance with another aspect hereof, the present disclosure provides, in at least one embodiment, an organ preservation system comprising a circuitous perfusion fluid system operable to perfuse a perfusion fluid through an organ, the preservation system comprising a fluid reservoir to support an organ, the fluid reservoir in fluid connection with a continuous or semi-continuous supply assembly for supplying a perfusion fluid or an excipient to the fluid reservoir, the fluid reservoir further in fluid connection with a separation device permitting the filtration of the perfusion fluid upon removal of the perfusion fluid from the fluid reservoir to separate and remove spent constituents from the perfusion fluid, and a pump for circulating the perfusion fluid through the circuitous fluid system.

In at least one embodiment, the organ preservation system can be used for perfusion with a hemoglobin based perfusion fluid.

In at least one embodiment, the organ preservation system can be used for perfusion with a red blood cell based perfusion fluid.

In at least one embodiment, the organ preservation system, can be used for perfusion with a finished oxygenation medium of the present disclosure.

Other features and advantages or the present disclosure will become apparent from the following detailed description. It should be understood, however, that the detailed description, while indicating preferred implementations of the present disclosure, is given by way of illustration only, since various changes and modification within the spirit and scope of the disclosure will become apparent to those of skill in the art from the detailed description.

### DETAILED DESCRIPTION

Various processes, methods and compositions will be described below to provide an example of an embodiment of the claimed subject matter. No embodiment described below limits any claimed subject matter and any claimed subject matter may cover processes, methods, or compositions that differ from those described below. The claimed subject matter is not limited to any process, method, or composition having all of the features of processes, methods, or compositions described below, or to features common to multiple or processes, methods, compositions or compositions described below. It is possible that a process, method, or composition described below is not an embodiment of any claimed subject matter. Any subject matter disclosed in processes, methods, or compositions described below that is not claimed in this document may be the subject matter of another protective instrument, for example, a continuing patent application, and the applicants, inventors or owners do not intend to abandon, disclaim or dedicate to the public any such subject matter by its disclosure in this document.

As used herein and in the claims, the singular forms, such “a”, “an” and “the” include the plural reference and vice versa unless the context clearly indicates otherwise. Throughout this specification, unless otherwise indicated, “comprise,” “comprises” and “comprising” are used inclusively rather than exclusively, so that a stated integer or group of integers may include one or more other non-stated integers or groups of integers. The term “or” is inclusive unless modified, for example, by “either”. The term “and/or” is intended to represent an inclusive or. That is “X and/or Y” is intended to mean X or Y or bot, for example. As a further example, X, Y and/or Z is intended to mean X or Y or Z or any combination thereof.

When ranges are used herein for physical properties, such as molecular weight, or chemical properties, such as chemical formulae, all combinations and sub-combinations of ranges and specific embodiments therein are intended to be included. Other than in the operating examples, or where otherwise indicated, all numbers expressing quantities of ingredients or reaction conditions used herein should be understood as modified in all instances by the term “about.” The term “about” when referring to a number or a numerical range means that the number or numerical range referred to is an approximation within experimental variability (or within statistical experimental error), and thus the number or numerical range may vary between 1% and 15% of the stated number or numerical range, as will be readily recognized by context. Furthermore, any range of values described herein is intended to specifically include the limiting values of the range, and any intermediate value or sub-range within the given range, and all such intermediate values and sub-ranges are individually and specifically disclosed (e.g. a range of 1 to 5 includes 1, 1.5, 2, 2.75, 3, 3.90, 4, and 5). Similarly, other terms of degree such as “substantially” and “approximately” as used herein mean a reasonable amount of deviation of the modified term such that the end result is not significantly changed. These terms of degree should be construed as including a deviation of the modified term if this deviation would not negate the meaning of the term it modifies.

Unless otherwise defined, scientific and technical terms used in connection with the formulations described herein shall have the meanings that are commonly understood by those of ordinary skill in the art. The terminology used herein is for the purpose of describing particular embodiments only, and is not intended to limit the scope of the present invention, which is defined solely by the claims.

All publications, patents, and patent applications referred herein are herein incorporated by reference in their entirety to the same extent as if each individual publication, patent or patent application was specifically indicated to be incorporated by reference in its entirety.

### Definitions

The terms “red blood cell” or “erythrocyte”, as may be used interchangeably herein, mean a non-nucleated cell containing hemoglobin that circulates in blood, generally responsible for the red color of blood.

The term “ex-vivo”, as used herein is to denote outside a human or animal body such that an organ, tissue or cells are wholly or partially separated from the human or animal's normal blood circulation.

By “formulating the oxygenation constituent to form a finished oxygenation medium” it is meant that the oxygenation constituent is contacted with at least one other ingredient, including, but not limited to, a diluent or excipient and mixed, homogenized or prepared until a finished oxygenation medium is formed.

The term “finished oxygenation medium”, as used herein, refers to a fully formulated oxygenation medium comprising an oxygenation constituent suitable for ex-vivo use.

The terms “HBOC”, or “hemoglobin based oxygen carrier”, as used herein, refer to an oxygen carrying hemoglobin derivative wherein the hemoglobin molecule has been modified, for example by chemically cross-linking of hemoglobin molecules, polymerization, or by conjugation of hemoglobin to other molecules, for example, conjugation to polyethylene glycol (PEG) or dextran.

The terms “hemoglobin” and “hemoglobin molecule”, as used interchangeably herein, refer to the protein contained within erythrocytes that transports oxygen in living organisms. Each molecule of hemoglobin has 4 subunits, 2 α-chains and 2 β-chains, which are arranged in a tetrameric structure. Each subunit also contains one heme group, which is the iron-containing center that binds oxygen. Thus, each hemoglobin molecule can bind 4 oxygen molecules. As used herein, the term by itself refers to native hemoglobin, including naturally occurring variants thereof, and further includes hemoglobin obtainable from any invertebrate or vertebrate animal, or a human, including, without limitation, e.g. human hemoglobin, bovine hemoglobin, ovine hemoglobin, and porcine hemoglobin.

“Substantially pure”, as used herein, herein describes an entity, e.g. a cell or chemical or biochemical compound, such as hemoglobin, which has been separated from constituents that naturally accompany it. Typically, an entity is substantially pure when at least 60%, more preferably at least 75%, more preferably at least 90%, 95%, 96%, 97%, or 98%, and most preferably at least 99% of the total material (by volume, by wet or dry weight, or by mole percent or mole fraction) in a sample is the entity of interest. With reference to an erythrocyte preparation, the term further refers to the separation from of non-erythrocyte blood cells, e.g. leukocytes and thrombocytes including a preparation comprising no more than 5%, 4%, 3%, 2% or 1% of non-erythrocyte blood cells. Purity can be measured by any appropriate method, e.g., in the case of proteins, by chromatography, gel electrophoresis or HPLC analysis, and in the case of red blood cells, flow cytometry.

### General Implementation

In overview, it has surprisingly been realized, that preparations comprising non-modified hemoglobin, HBOC, or a mixture thereof when mixed with certain quantities of red blood cells can be used as oxygenation constituents. In particular, hemoglobin preparations comprising from about 10% to about 99% by weight hemoglobin of the oxygenation constituents, can be mixed with red blood cell preparations comprising the balance by weight hemoglobin of the oxygenation constituents. The novel oxygenation constituents can be used to prepare finished oxygenation media. The finished oxygenation media are suitable to provide tissue or organ oxygenation and thereby mediate ex-vivo preservation of organs and tissues. Surprisingly, only small amounts of methemoglobin are formed in the finished oxygenation media of the present disclosure, thus allowing for prolonged oxygenation and preservation of organs or tissues. Furthermore, the oxygenation constituents and the finished oxygenation media of the present disclosure exhibit limited red blood cell lysis, and limited coagulation. The finished oxygenation media of the present disclosure permit the ex-vivo preservation of organs and tissues for extended periods of time, for example, for at least about 6 hours, under static conditions, or for a period of at least 12 hours using a machine perfusion system. A yet further significant benefit of the finished oxygenation media of the present disclosure is that they can be used within a wide temperature range. It is, for example, possible to use the finished oxygenation media at a temperature as low as about 2° C., and as high as about 40° C. The oxygenation constituents and finished oxygenation media can be safely and economically manufactured and methods for manufacture are also included herein. Thus, the disclosure provides novel oxygenation constituents which can be used to prepare finished oxygenation media for ex-vivo use, for example, during surgical procedures, in order to preserve tissues and organs for transplantation, and for other uses.

In what follows selected embodiments are described. Accordingly, the present disclosure provides, in one embodiment, an oxygenation constituent for formulating a finished oxygenation medium, the oxygenation constituent comprising a mixture of a first and second preparation wherein:
(i) the first preparation is a hemoglobin preparation comprising non-modified hemoglobin, HBOC, or a mixture thereof in an amount of from about 1% to about 40% by weight hemoglobin, the balance of the hemoglobin preparation comprising an aqueous hemoglobin diluent; and
(ii) the second preparation is a red blood cell preparation comprising hemoglobin in an amount of from about 1% to about 40% by weight hemoglobin, and the balance of the red blood cell preparation comprising an aqueous red blood cell diluent; and
wherein
   the mixture is formed such that the oxygenation constituent comprises an amount of from about 10% to about 99% by weight hemoglobin of the hemoglobin preparation, and an amount equal to the balance by weight hemoglobin of the red blood cell preparation.

In general, the oxygenation constituents can be prepared by initially providing a hemoglobin preparation or HBOC preparation or a mixture thereof, and a red blood cell preparation.

In an embodiment, red blood cells, hemoglobin and HBOC preparations can be manufactured using whole blood as a source. Suitable blood that may be used in accordance herewith includes invertebrate or vertebrate animal or human blood, including, without limitation, mammalian and avian blood, including without limitation, human blood, bovine blood, porcine blood, equine blood and ovine blood. Blood solutions may be collected from live or dead organisms and may be collected using any techniques and devices known to the art, including, for example, the methodologies described in U.S. Pat. Nos. 5,084,558 and 5,296,465. The blood may be fresh or from an older sample, for example expired blood from a blood bank institute. In addition, the blood may have been stored and/or frozen. It is preferred that the blood solution is screened for the presence of blood borne pathogens, for example where human blood is used, HIV and hepatitis B. Preferably, blood free of blood borne pathogens is selected for use in accordance with the methods of the present disclosure. Upon blood collection, it is preferred that an anti-coagulant is added to the blood to prevent blood clotting. Anti-coagulants that may be used include heparin, hirudin, sodium citrate, ethylenediaminetetraacetic acid. The anticoagulant may be provided as an aqueous solution or in particulate form.

In order to isolate red blood cells from blood, any red blood cell separation technique known to the art may be used. This includes the use of centrifugation and/or straining and filtering techniques to remove large blood aggregates (e.g. 50 μm and larger) and debris. Thus, one or more propylene 800 μm to 50 μm filters may be used. It is noted in this regard that red blood cells are about 5-10 μm in size.

In some embodiments, the red blood cells can be further isolated from blood by diafiltration using an isotonic solution, having a pH and osmolarity which preserves the integrity of the erythrocyte cellular membrane, for example, a sodium citrate (about 6.0 g/l) and sodium chloride (about 8.0 g/l) solution having an osmolarity of 285-315 mOsm. Acceptable diafiltration filters that can be used in accordance herewith include microporous membranes which substantially separate erythrocytes from smaller components for example, a modified polyethersulfone hollow fiber tangential flow filtration membrane obtainable from Spectrum labs. The isotonic solution can be added in batches or continuously, typically approximately at the same rate at which filtrate is lost. During this step, components of the blood solution smaller than red blood cells, generally the plasma portion of the blood, including extracellular blood proteins, e.g. antibodies and serum albumins, are separated as filtrate from the red blood cells, which are retained and continuously or batch-wise added to the isotonic solution. Volumes of isotonic solution used may vary and may for example be at least 2×, 3×, 4×, 5×, 6× or 7× the volume of blood solution. In some embodiments, sufficient volumes of isotonic solution are used to remove at least from about 90% to 95% (mole/mole) of blood plasma proteins and obtain a substantially pure red blood cell preparation, for example a red blood cell preparation that is at least about 90%, at least about 95%, or at least about 99% pure.

The techniques used to isolate red blood cells from blood and to obtain a substantially pure red blood cell preparation may be as desired, and include any techniques known to the art, including, for example, the methods for isolating erythrocytes are described in U.S. Pat. No. 5,955,581.

In order to prepare a red blood cell preparation comprising of from about 1% to about 40% by weight hemoglobin, the red blood cell preparation can be mixed with a physiologically compatible aqueous red blood cell diluent, i.e. an aqueous diluent in which the red blood cells remain substantially intact, and in which the red blood cells substantially retain their physiological oxygen carrying capabilities. Thus, for example, the red blood cell preparation can be diluted using a saline solution, or a lactated Ringer's solution, or a modified lactated Ringer's solution. The amount of aqueous red blood cell diluent can vary, and can, for example, be selected so that the fully diluted red blood cell preparation comprises hemoglobin in an amount of from about 1% to about 40% by weight hemoglobin, and the balance of the red blood cell preparation comprising an aqueous red blood cell diluent; or from about 5% to about 35% by weight hemoglobin, and the balance of the red blood cell preparation comprising an aqueous red blood cell diluent; or from about 10% to about 30% by weight hemoglobin, and the balance of the red blood cell preparation comprising an aqueous red blood cell diluent; or from about 15% to about 25% by weight hemoglobin, and the balance of the red blood cell preparation comprising an aqueous red blood cell diluent; or from about 20% by weight hemoglobin, and the balance of the red blood cell preparation comprising an aqueous red blood cell diluent.

Turning now to hemoglobin and HBOCs, in one embodiment, isolated red blood cells may be used as a source whence hemoglobin can be extracted to obtain an isolated hemoglobin preparation. Thus, for example, isolated red blood cells can be lysed. Any technique for lysing erythrocytes known to the art may be used, including any mechanical lysis technique or chemical lysis technique, provided however that such technique does not substantially negatively affect the ability of hemoglobin to transport and release oxygen.

In one embodiment, the isolated red blood cells can be lysed by subjecting the red blood cells to a hypotonic shock to obtain a red blood cell lysate. Suitable hypotonic solutions that may be used in accordance herewith include, for example, a phosphate buffer 3.75 mM, pH 7.2 or water which may be mixed with the red blood cells, and the mixture may be incubated on ice, for example for 1 hour to obtain a lysed red blood cell preparation. Subsequently a hemoglobin fraction can be obtained from the red blood cell lysate using a variety of suitable protein purification techniques, including chromatographic separation techniques which include absorption based methods, involving initial retention of hemoglobin on solid chromatographic media, and thereafter solvent based elution, or flow-through based techniques, involving retention of impurities and flow through of hemoglobin. Other chromatography based techniques include affinity chromatography and high performance liquid based chromatography (HPLC). Chromatographic techniques to isolate hemoglobin and obtain an isolated hemoglobin preparation that can be used further include, for example, those described in U.S. Pat. No. 5,691,453, Andrade et al., Int. J. Biol. Macromol, 2004, 34: 233-240, and Lu et al., Artif. Cells Blood Substit. Immobil. Biotechnol. 2004, 32: 2004. Alternatively, in order to isolate hemoglobin, the red blood cell lysates may be subjected non-chromatography based purification techniques, including, for example, aqueous phase extraction, membrane based micro- and ultrafiltration techniques and tangential flow based techniques. Ultrafiltration based techniques that can be used include those described by Feins et al. 2005, J. Membr. Sci 248: 137-148. Tangential flow based techniques that can be used include those described for example by Palmer et al., 2009, in Biotechnol. Progr, 2009, 26 (1) 189-199 and Elmer et al., Biotechnol. Progr. 2009, 25(5) 1402-1410. Two-phase aqueous extraction techniques that can be used to obtain hemoglobin include those described in U.S. Pat. No. 5,407,579.

Using the foregoing techniques, or combinations or modifications thereof, or other suitable techniques, in some embodiments substantially pure hemoglobin preparations can be obtained, including, for example, preparations which are at least about 80%, 85%, 90%, 95%, 96%, 97%, 98%, or 99% pure.

The hemoglobin obtained using the foregoing techniques, or combinations or modifications thereof, or other suitable techniques is non-modified hemoglobin, i.e. hemoglobin which has not been chemically modified.

In some embodiments, the hemoglobin obtained in accordance with the foregoing techniques, combinations or modifications thereof can be used to prepare hemoglobin preparations comprising non-modified hemoglobin in accordance herewith. In order to prepare a hemoglobin preparation comprising non-modified hemoglobin, non-modified hemoglobin can be mixed with a physiologically compatible aqueous hemoglobin diluent. Thus, for example, hemoglobin can be diluted using a saline solution, or a lactated Ringer's solution, or a modified lactated Ringer's solution. The amount of aqueous hemoglobin diluent can vary, and can, for example, be selected so that the fully diluted hemoglobin preparation comprises non-modified hemoglobin in an amount of from about 1% to about 40% by weight hemoglobin, and the balance of the hemoglobin preparation comprising the aqueous hemoglobin diluent; or in an amount of from about 5% to about 35% by weight hemoglobin, and the balance of the hemoglobin preparation comprising the aqueous hemoglobin diluent; or in an amount of from about 5% to about 35% by weight hemoglobin; or in an amount of from about 10% to about 30% by weight hemoglobin, and the balance of the hemoglobin preparation comprising the aqueous hemoglobin diluent; or in an amount of from about 15% to about 25% by weight hemoglobin, and the balance of the hemoglobin preparation comprising the aqueous hemoglobin diluent. The foregoing hemoglobin preparations are substantially free of modified hemoglobin.

In some embodiments, a hemoglobin formulating agent can be included to prepare the hemoglobin preparation, for example, a formulating agent to encapsulate isolated hemoglobin in liposomes, and form liposome encapsulated hemoglobin (LEH), see for example: U.S. Patent Application No. 2014/0212477.

The hemoglobin obtained from red blood cells can also be used to prepare HBOC preparations, including polymerized HBOC preparations, for example, by cross-linking of hemoglobin molecules, or by chemically modifying hemoglobin molecules. Examples of polymerized hemoglobin molecules are described in U.S. Pat. Nos. 5,955,581; 5,895,810; U.S. Patent Application US2012/0028899 and PCT Patent Application PCT/CA2017/051111. Examples of chemically modified hemoglobin molecules include polysaccharide conjugated hemoglobin (e.g. PCT Patent Application PCT/CA99/00260), polyalkylene oxide-conjugated hemoglobin (e.g. U.S. Pat. No. 5,650,388) and polyethylene glycol (PEG) conjugated hemoglobin (e.g. U.S. Pat. Nos. 5,750,725, 7,144,989 and 8,609,815).

In some embodiments, the HBOC preparation obtained in accordance with the foregoing techniques, combinations or modifications thereof can be used to prepare hemoglobin preparations comprising HBOC in accordance herewith. In order to prepare a hemoglobin preparation comprising HBOC, the HBOC preparation can be mixed with a physiologically compatible aqueous hemoglobin diluent. Thus, for example, the HBOC preparation can be diluted using a saline solution, or a lactated Ringer's solution, or a modified lactated Ringer's solution. The amount of aqueous hemoglobin diluent can vary, and can, for example, be selected so that the fully diluted hemoglobin preparation comprises HBOC in an amount of from about 1% to about 40% by weight hemoglobin, and the balance of the hemoglobin preparation comprising the aqueous hemoglobin diluent; or in an amount of from about 5% to about 35% by weight hemoglobin, and the balance of the hemoglobin preparation comprising the aqueous hemoglobin diluent; or in an amount of from about 5% to about 35% by weight hemoglobin; or in an amount of from about 10% to about 30% by weight hemoglobin, and the balance of the hemoglobin preparation comprising the aqueous hemoglobin diluent; or in an amount of from about 15% to about 25% by weight hemoglobin, and the balance of the hemoglobin preparation comprising the aqueous hemoglobin diluent. The foregoing HBOC preparations can be substantially free of non-modified hemoglobin. Thus the HBOC preparations can contain less 10% (w/w), less than 5% (w/w), or less then 1% (w/w) of non-modified hemoglobin.

In some embodiments, a mixture of non-modified hemoglobin and HBOC can be used to prepare a hemoglobin preparation. Such a mixture can be prepared by obtaining a hemoglobin preparation comprising non-modified hemoglobin and an HBOC preparation, using techniques or combinations or modifications thereof as is hereinbefore described, and contacting and mixing the two preparations in order to obtain a substantially homogenous mixture of non-modified hemoglobin and HBOC. The relative amounts of HBOC preparation and non-modified hemoglobin preparation within the mixture may be varied, and selected as desired. Thus, for example, the preparations comprising a mixture of non-modified hemoglobin and HBOC can be selected so that the fully diluted hemoglobin preparation comprises HBOC and non-modified hemoglobin together in an amount of from about 1% to about 40% by weight hemoglobin, and the balance of the hemoglobin preparation comprising the aqueous hemoglobin diluent; or in an amount of from about 5% to about 35% by weight hemoglobin, and the balance of the hemoglobin preparation comprising the aqueous hemoglobin diluent; or in an amount of from about 5% to about 35% by weight hemoglobin; or in an amount of from about 10% to about 30% by weight hemoglobin, and the balance of the hemoglobin preparation comprising the aqueous hemoglobin diluent; or in an amount of from about 15% to about 25% by weight hemoglobin, and the balance of the hemoglobin preparation comprising the aqueous hemoglobin diluent.

Red blood cell preparations and hemoglobin preparations can also be obtained from a commercial supplier. Commercial hemoglobin suppliers include Thomas Scientific and Sigma Aldrich. Commercial red blood cell suppliers include blood banks such as the Red Cross.

Having obtained a preparation comprising non-modified hemoglobin, HBOC, or a mixture thereof, and a red blood cell preparation, the two preparations can be contacted and mixed until a substantially homogenous mixture is obtained. The amounts of hemoglobin and red blood cell preparation can be varied.

In one embodiment, the oxygenation constituent can be prepared to constitute an amount of the hemoglobin preparation so that the hemoglobin preparation comprises from about 10% to about 99% by weight hemoglobin of the oxygenation constituent, and an amount of the red blood cell preparation so that the balance by weight hemoglobin to 100% (i.e. from about 1% to about 90%) of the oxygenation constituent constitutes the red blood cell preparation.

In one embodiment, the oxygenation constituent can be prepared to constitute an amount of the hemoglobin preparation so that the hemoglobin preparation comprises from about 20% to about 90% by weight hemoglobin of the oxygenation constituent, and an amount of the red blood cell preparation so that the balance by weight hemoglobin to 100% (i.e. from about 10% to about 80%) of the oxygenation constituent constitutes the red blood cell preparation.

In one embodiment, the oxygenation constituent can be prepared to constitute an amount of the hemoglobin preparation so that the hemoglobin preparation comprises from about 30% to about 80% by weight hemoglobin of the oxygenation constituent, and an amount of the red blood cell preparation so that the balance by weight hemoglobin to 100% (i.e. from about 20% to about 70%) of the oxygenation constituent constitutes the red blood cell preparation.

In one embodiment, the oxygenation constituent can be prepared to constitute an amount of the hemoglobin preparation so that the hemoglobin preparation comprises from about 40% to about 70% by weight hemoglobin of the oxygenation constituent, and an amount of the red blood cell preparation so that the balance by weight hemoglobin to 100% (i.e. from about 30% to about 60%) of the oxygenation constituent constitutes the red blood cell preparation.

In one embodiment, the oxygenation constituent can be prepared to constitute an amount of the hemoglobin preparation so that the hemoglobin preparation comprises from about 50% to about 60% by weight hemoglobin of the oxygenation constituent, and an amount of the red blood cell preparation so that the balance by weight hemoglobin to 100% (i.e. from about 40% to about 50%) of the oxygenation constituent constitutes the red blood cell preparation.

In one example embodiment, the present disclosure includes an oxygenation constituent for formulating a finished oxygenation medium, the oxygenation constituent comprising a mixture of a first and second preparation wherein:
(i) the first preparation is a hemoglobin preparation comprising non-modified hemoglobin, HBOC or a mixture thereof in an amount of from about 5% to about 15% by weight hemoglobin, the balance of the hemoglobin preparation comprising an aqueous hemoglobin diluent; and
(ii) the second preparation is a red blood cell preparation comprising hemoglobin in an amount of from about 10% to about 30% by weight hemoglobin, and the balance of the red blood cell preparation comprising an aqueous red blood cell diluent; and wherein
   the mixture is formed such that the oxygenation constituent comprises an amount from about 40% to about 60% by weight hemoglobin of the hemoglobin preparation, and an amount equal to the balance by weight hemoglobin of the red blood cell preparation.

In one example embodiment, the oxygenation constituent can comprise an oxygenation constituent for formulating a finished oxygenation medium, the oxygenation constituent comprising a mixture of a first and second preparation wherein:
(i) the first preparation is a hemoglobin preparation comprising non-modified hemoglobin, HBOC or a mixture thereof in an amount of about 10% by weight hemoglobin, the balance of the hemoglobin preparation comprising an aqueous hemoglobin diluent; and
(ii) the second preparation is a red blood cell preparation comprising hemoglobin in an amount of about 20% by weight hemoglobin, and the balance of the red blood cell preparation comprising an aqueous red blood cell diluent; and
wherein
   the mixture is formed such that the oxygenation constituent comprises an amount of about 50% by weight hemoglobin of the hemoglobin preparation, and an amount equal the balance by weight hemoglobin of the red blood cell preparation.

The thus obtained mixture can be used as an oxygenation constituent to formulate a finished oxygenation medium.

To briefly recap, a non-modified hemoglobin preparation or, as the case may be, an HBOC preparation or a preparation containing a mixture of non-modified hemoglobin and HBOC, is provided, and separately a red blood cell preparation is provided. The preparation comprising non-modified hemoglobin, HBOC, or a mixture thereof and the red blood cell preparation are mixed to obtain an oxygenation constituent.

In one embodiment, the oxygenation constituent can be contacted with at least one other ingredient suitable for use in finished oxygenation, notably a diluent or excipient. Thus, in another aspect, the present disclosure provides a finished oxygenation medium comprising:
(a) from about 1% by weight hemoglobin to about 39% by weight hemoglobin of an oxygenation constituent, the oxygenation constituent comprising a mixture of a first and second preparation wherein:
   (i) the first preparation is a hemoglobin preparation comprising non-modified hemoglobin, HBOC, or a mixture thereof in an amount of from about 1% to about 40% by weight hemoglobin, the balance of the hemoglobin preparation comprising an aqueous hemoglobin diluent; and
   (ii) the second preparation is a red blood cell preparation comprising hemoglobin in an amount of from about 1% to about 40% by weight hemoglobin, and the balance of the red blood cell preparation comprising an aqueous red blood cell diluent; and
   wherein
      the mixture is formed such that the oxygenation constituent comprises an amount of from about 10% to about 99% by weight hemoglobin of the hemoglobin preparation, and an amount equal to the balance by weight hemoglobin of the red blood cell preparation; and
(b) the balance of the medium comprising at least one other formulary ingredient selected from a diluent or an excipient, and optionally other ingredients suitable for formulating a finished oxygenation medium.

The present disclosure further provides, in at least one embodiment, a use of a use of an oxygenation constituent to formulate a finished oxygenation medium, the oxygenation constituent comprising a mixture of a first and second preparation wherein:
(i) the first preparation is a hemoglobin preparation comprising non-modified hemoglobin, HBOC, or a mixture thereof in an amount of from about 1% to about 40% by weight hemoglobin, the balance of the hemoglobin preparation comprising an aqueous hemoglobin diluent; and
(ii) the second preparation is a red blood cell preparation comprising hemoglobin in an amount of from about 1% to about 40% by weight hemoglobin, and the balance of the red blood cell preparation comprising an aqueous red blood cell diluent; and
wherein
   the mixture is formed such that the oxygenation constituent comprises an amount of from about 10% to about 99% by weight hemoglobin of the hemoglobin preparation, and an amount equal to the balance by weight hemoglobin of the red blood cell preparation.

The oxygenation constituent and diluent or excipient are mixed, homogenized or prepared, preferably until a homogenous mixture of the diluent, or excipient and oxygenation constituent is formed, wherein such mixture is suitable for use as an oxygenation medium. The diluent or excipient may be any suitable diluent or excipient, and the diluent or excipient may be provided in any form, including, for example, as a solution, suspension, gel, or liquid. Diluents that can be included include buffers, such as isotonic saline, 2-(N-morpholino)ethanesulfonic acid (MES), and excipients that can be used include lactated Ringer's Solution.

Optionally, a plurality of other ingredients can be provided, for example at least 2, 3, 4, 5, 6, 7, 8, 9, 10 or more ingredients, in addition to the oxygenation constituent and diluent or excipient, to prepare the finished oxygenation medium. In embodiments hereof which include a plurality of ingredients, such ingredients may be mixed sequentially or simultaneously.

In one embodiment, the additional ingredients can include compounds normally found in blood, for example, ions normally found in blood, including calcium ions; chloride ions; sodium ions; magnesium ions; phosphate ions; or mixtures thereof, each of which can be provided in a variety of chemical forms, including, for example sodium chloride, and a variety of formulations, for example in the form of a saline solution. Additional other ingredients that can be included to prepare a finished oxygenation medium are buffering agents, for example 2-(N-morpholino)ethanesulfonic acid (MES); redox agents, for example sulfhydryls, such as glutathione, ascorbic acid (ascorbate), n-acetyl cysteine, nicotinamide adenine dinucleotide phosphate (NADH), reduced nicotinamide adenine dinucleotide phosphate (NADPH); colloids, such as hydroxyethyl starch or albumin; sugars, including monosaccharides or disaccharides, such as glucose or lactobionic acid; amino acids; agents to limit the presence of oxygen radicals, including superoxide dismutase; agents to limit the formation of methemoglobin, methemoglobin reductase, for example; antibiotics; or mixtures thereof. In some embodiments, even pharmacological compounds capable of ameliorating a disease state or medical condition can be included.

The quantities of the other ingredients, including the excipient or diluent can vary, however typically the oxygenation constituent constitutes at least 1% by weight of hemoglobin of the finished oxygenation medium.

In one embodiment, the finished oxygenation medium can comprise from about 1% by weight hemoglobin to about 39% by weight hemoglobin of the oxygenation constituent.

In one embodiment, the finished oxygenation medium can comprise from about 5% by weight hemoglobin to about 35% by weight hemoglobin of the oxygenation constituent.

In one embodiment, the finished oxygenation medium can comprise from about 10% by weight hemoglobin to about 30% by weight hemoglobin of the oxygenation constituent.

In one example embodiment, the finished oxygenation medium can comprise from about 15% by weight hemoglobin to about 25% by weight hemoglobin of an oxygenation constituent, the oxygenation constituent comprising:
(i) a hemoglobin preparation comprising non-modified hemoglobin, HBOC or a mixture thereof in an amount of from about 5% to about 15% by weight hemoglobin, the balance of the hemoglobin preparation comprising an aqueous hemoglobin diluent; and
(ii) a red blood cell preparation comprising hemoglobin in an amount of from about 10% to about 30% by weight hemoglobin, and the balance of the red blood cell preparation comprising an aqueous red blood cell diluent; and
wherein
   the mixture is formed such that the oxygenation constituent comprises an amount from about 40% to about 60% by weight hemoglobin of the hemoglobin preparation, and an amount equal to the balance by weight hemoglobin of the red blood cell preparation.

In one example embodiment, the finished oxygenation medium can comprise about 20% by weight hemoglobin of an oxygenation constituent, the oxygenation constituent comprising:
(i) a hemoglobin preparation comprising non-modified hemoglobin, HBOC or a mixture thereof in an amount of about 10% by weight hemoglobin, the balance of the hemoglobin preparation comprising an aqueous hemoglobin diluent; and
(ii) a red blood cell preparation comprising hemoglobin in an amount of about 20% by weight hemoglobin, and the balance of the red blood cell preparation comprising an aqueous red blood cell diluent; and
wherein
   the mixture is formed such that the oxygenation constituent comprises an amount of about 50% by weight hemoglobin of the hemoglobin preparation, and an amount equal the balance by weight hemoglobin of the red blood cell preparation.

Thus in accordance with the foregoing methods a finished oxygenation can be prepared. Accordingly, the present disclosure includes, in a further embodiment, a method of preparing a finished oxygenation medium for ex-vivo use, the method comprising:
(i) providing a hemoglobin preparation comprising non-modified hemoglobin, HBOC, or a mixture thereof in an amount of from about 1% to about 40% by weight hemoglobin, the balance of the hemoglobin preparation comprising an aqueous hemoglobin diluent;
(ii) providing a red blood cell preparation comprising hemoglobin in an amount of from about 1% to about 40% by weight hemoglobin, and the balance of the red blood cell preparation comprising an aqueous red blood cell diluent;
(iii) mixing the hemoglobin preparation and the red blood cell preparation to form an oxygenation constituent such that the oxygenation constituent comprises an amount of from about 10% to about 99% by weight hemoglobin of the hemoglobin preparation, and an amount equal to the balance by weight hemoglobin of the red blood cell preparation; and
(iv) formulating the oxygenation constituent with at least one formulary ingredient selected from an excipient, and a diluent, and optionally other ingredients suitable for formulating a finished oxygenation medium.

In one embodiment, the finished oxygenation media may be stored for shorter or longer periods of time, for example, from 1-2 days up to 1 year or more. Finished oxygenation media are preferably stored in sterile, sealed containers, for example, sealed glass containers, stainless steel containers or storage bags, having a low oxygen environment. Storage containers are further preferably impermeable to the transfer of water in order to prevent evaporation of water and concentration of the media. In order to achieve a low oxygen environment storage containers may be blanketed with, for example, a nitrogen atmosphere prior to sealing. In some embodiments, in order to prevent auto-oxidation, i.e. the formation of methemoglobin, the finished oxygenation medium can be treated with carbon monoxide. In order to store the oxygenation media, the containers can be refrigerated (0° C. to 4° C.) for storage, or the media may be frozen and stored in a freezer, for example from −20° C. to −80° C.

In a further aspect, the present disclosure provides, in one embodiment,

a use of a finished oxygenation medium for the ex-vivo preservation of a tissue or an organ, the finished oxygenation medium comprising:
(a) from about 1% by weight hemoglobin to about 39% by weight hemoglobin of an oxygenation constituent, the oxygenation constituent comprising a mixture of a first and second preparation wherein:
   (i) the first preparation is a hemoglobin preparation comprising non-modified hemoglobin, HBOC, or a mixture thereof in an amount of from about 1% to about 40% by weight hemoglobin, the balance of the hemoglobin preparation comprising an aqueous hemoglobin diluent; and
   (ii) the second preparation is a red blood cell preparation comprising hemoglobin in an amount of from about 1% to about 40% by weight hemoglobin, and the balance of the red blood cell preparation comprising an aqueous red blood cell diluent; and
   wherein
      the mixture is formed such that the oxygenation constituent comprises an amount of from about 10% to about 99% by weight hemoglobin of the hemoglobin preparation, and an amount equal to the balance by weight hemoglobin of the red blood cell preparation; and
(b) the balance of the medium comprising at least one other formulary ingredient selected from a diluent or an excipient, and optionally other ingredients suitable for formulating a finished oxygenation medium.

In one embodiment, the finished oxygenation medium of the present disclosure can be used to maintain the oxygen content of an organ or tissue ex-vivo in order to preserve an organ, for example, when organs are stored for later transplantation to a patient, or for reimplantation in a patient, or when organs require transportation. Organ transplants may be autotransplants (i.e. transplantation of an organ from one part of the body to another part of the body of the same subject), allotransplants (i.e. transplantation of an organ from one subject to another subject), or xenotransplants (i.e. transplantation of an organ from a subject of one species to a subject of another species). Individual organs that may be used in this regard include, without limitation liver, kidney, heart, lung, intestine and pancreas. The organ can be in any stage of development or life, or of any age, or of any origin including a human organ, or an organ obtainable from another vertebrate.

In one embodiment, the finished oxygenation medium of the present disclosure can be used to maintain the oxygen content of a tissue ex-vivo in order to preserve a tissue, or for tissue engineering. Thus tissue can be preserved, for example, when tissue is stored for later transplantation or grafting to a patient, or for reimplantation in a patient, or when tissue requires transportation, or when tissue is used for experimental ex vivo purposes, for example, the study of cancer tumors and treatment thereof. Tissue grafts may be autografts (i.e. transplantation of tissue from one part of the body to another part of the body of the same subject), allografts (i.e. transplantation of tissue from one subject to another subject), or xenografts (i.e. transplantation of tissue from a subject of one species to a subject of another species). Further examples of tissue transplants include composite tissue allotransplants, e.g. limbs, face.

In one embodiment, the finished oxygenation media of the present disclosure can be used to maintain organs for research and development purposes, for example, for the discovery of biomarkers, for tissue engineering or biofabrication, of for use of organ tissue as a bioreactor.

In one embodiment, the organs or tissue can be preserved using a preservation system permitting preservation in static mode. In other embodiments, the organs or tissue can be preserved using a preservation system permitting preservation in dynamic mode. In static mode, the organs are bathed in a solution comprising the oxygenation medium of the present disclosure. In dynamic mode, the organ is perfused using the finished oxygenation medium of the present disclosure and one or more mechanical devices including, for example, a pump system, or devices for regulating temperature, i.e. a machine perfusion system. Various preservation systems are known to the art and include, by way of example, the systems described in PCT Patent Applications: PCT/US2013/049573 and PCT/US2016/051122, and US Patent Applications: U.S. 2008/0038811 and U.S. 2010/0304352.

In some embodiments, the preservation system can be configured to include a separation device. In these embodiments, separation devices allow for removal of certain constituents from the finished oxygenation medium, including non-modified hemoglobin, HBOCs or red blood cells, or any of the additional ingredients, and replacement with fresh constituents. Thus, for example, a preservation system can be configured to include a continuous or semi-continuous supply assembly for a finished oxygenation medium or an excipient, a separation device, and a pump, and further optionally an oxygenation cartridge. The preservation system can be operated to supply oxygenation medium or excipient on a continuous or semi-continuous basis, and filter the finished oxygenation medium or excipient, and replace it with fresh oxygenation medium or excipient from the supply assembly, thus allowing for continuous or semi-continuous refreshment of the excipient or finished oxygenation medium. Separation devices that may be used include filters which may be operated in line on a substantially continuous basis, using, for example, a tangential flow system, permitting continuous refreshment of the oxygenation medium or excipient, or filters which may be installed for use on a periodic basis. As will be appreciated by those in the art, filters and pore size of the filters may be selected in order to facilitate the selective removal of certain constituents of the finished oxygenation medium.

The present disclosure further includes in one embodiment, an organ preservation system comprising a circuitous perfusion fluid system operable to perfuse a perfusion fluid through an organ, the preservation system comprising a fluid reservoir to support an organ, the fluid reservoir in fluid connection with a continuous or semi-continuous supply assembly for supplying a perfusion fluid or an excipient to the fluid reservoir, the fluid reservoir further in fluid connection with a separation device permitting the filtration of the perfusion fluid upon removal of the perfusion fluid from the fluid reservoir, to separate and remove spent constituents from the perfusion fluid, and a pump for circulating the perfusion fluid through the circuitous fluid system. The system can further optionally contain an oxygenation cartridge. The organ preservation can be used in conjunction with the finished oxygenation media of the present disclosure, or in conjunction with any other perfusion fluid, for example, other hemoglobin based perfusion fluid or other red blood cell based perfusion fluids.

The period of time during which an organ or tissue may be preserved using the finished oxygenation medium of the present disclosure can vary. For example, an organ or tissue can be statically preserved for at least about 6 hours, at least about 8 hours, at least about 10 hours, or at least about 12 hours using a finished oxygenation medium of the present disclosure; or for example, an organ or tissue can be dynamically preserved for at least about 6 hours, at least about 12 hours, at least about 24 hours, at least about 48 hours, at least about 96 hours, or at least about 1 week, about 2 weeks, or about 4 weeks using a finished oxygenation medium of the present disclosure.

The finished oxygenation medium may be used at various temperatures ranging from as low as about 2° C., up to as high as about 40° C. to ex-vivo preserve organs or tissue. Other temperatures ranges at which the finished oxygenation medium may be used to ex-vivo preserve organs or tissue include temperatures from about 5° C. to about 35° C., from about 10° C. to about 30° C., and from about 15° C. to about 25° C. The finished oxygenation medium may further be used at about physiological temperature to ex-vivo preserve organs or tissue, i.e. at 37° C. for human tissue and organs, or at room temperature, i.e. from about 18° C. to about 23° C. The temperature may be varied in different circumstances, for example, depending on the specific organ or tissue that is being preserved, or the duration of the preservation time. Furthermore the temperature may be adjusted or optimized, for example by evaluation ex-vivo preservation at different temperatures or using different temperature regimens for different periods of time, and selected an optimized temperature.

The therapeutic amounts of the oxygenation medium used may vary. The term “therapeutically effective amount,” for the purposes of the present disclosure, refers to the amount of oxygenation medium which is effective to achieve its intended purpose, i.e. the sufficient ex-vivo oxygenation of an organ or tissue to survive for clinical use, or for clinical or medical research or development purposes. While individual situations may vary, determination of optimal ranges for effective amounts of a finished oxygenation medium to be used is within the skill of one in the art. Generally, dosages required to provide therapeutically effective amounts of the oxygenation medium or the oxygenation constituent, and which can be adjusted by one of ordinary skill in the art, will vary, depending on the tissue, organ, duration of preservation, health or physical condition of the tissue or organ, as can be determined by one of ordinary skill in the art using conventional considerations, (e.g. by means of an appropriate, conventional pharmacological or veterinary protocol).

As now can be appreciated, an oxygenation constituent and finished oxygenation medium can be prepared according to the methods of the present disclosure, which allows for prolonged periods of preservation of organs or tissues. The finished oxygen media can be applied in many ex-vivo clinical and clinical development processes.

Of course, the above described example embodiments of the present disclosure are intended to be illustrative only and in no way limiting. The described embodiments are susceptible to many modifications of composition, details and order of operation. The invention, rather, is intended to encompass all such modifications within its scope, as defined by the claims, which should be given a broad interpretation consistent with the description as a whole.

### EXAMPLES

Hereinafter are provided examples of further specific embodiments for performing the methods of the present disclosure, as well as embodiments representing the compositions of the present disclosure. The examples are provided for illustrative purposes only, and are not intended to limit the scope of the present disclosure in any way.

### Example 1—Preparation of an Oxygenation Constituent for Ex-Vivo Use

A low purity erythrocyte protein fraction consisting primarily of hemoglobin isolated by filtration was polymerized using glutaraldehyde and then reduced with cyanoborohydride and was put into a suitable buffer (lactated Ringer's with n-acetyl cysteine) such that the glutaraldehyde and cyanoborohidride were removed by filtration to thereby obtain an HBOC solution that was 11 g/dL (11%) by weight hemoglobin. The solution can either be end point sterilized with filtration or by irradiation. Packed red blood cells (pRBC), can be obtained commercially from one of numerous institutions dedicated to blood collection storage, and distribution, for example the Red Cross. An oxygenation constituent can be prepared by mixing, the HBOC solution and the pRBC in a 1 to 1. The oxygenation constituent can thereafter, as desired, immediately be used to prepare a finished oxygenation medium, for example as described in Example 2.

### Example 2—Preparation of a Finished Oxygenation Medium for Ex-Vivo Use

A finished oxygenation medium can be prepared using the oxygenation constituent as described in Example 1, and mixing the oxygenation constituent with a pre-packaged sterile isosmotic diluent containing saline, glucose, and an antibiotic. The oxygenation constituent and diluent can be mixed at room temperature prior to a final hemoglobin concentration of 10 g/dL (10%) by weight hemoglobin to obtain a finished oxygenation medium. The temperature of the finished oxygenation medium may be adjusted prior to ex-vivo use.

### Example 3—Ex-Vivo Use of a Finished Oxygenation Medium

An ex-vivo use of a finished oxygenation medium can include a freshly isolated human or animal organ, for example a kidney, canulated for fluid flow though the vasculature, suspended in a temperature controlled tank in the finished oxygenation medium as prepared in accordance with Examples 1 and 2 under best practices asceptic conditions. The finished oxygenation medium can be circulated through the organ and holding tank at appropriate flow and pressure settings to avoid damage to the organ. The medium can be kept oxygenated by use of an oxygenation cartridge in the circulation loop. If metabolism is desired the temperature can be controlled to physiologic norms. If just prolonged storage is desired, the temperature can be reduced. Glucose and metabolite levels can be monitored by use of existing analyzers and if levels are not appropriate, a diluent exchange can be initiated by diafiltration. The organ can be maintained until required for its intended use, for example transplantation, or until its condition is deemed to have deteriorated too far for its intended use.

1. An oxygenation constituent for formulating a finished oxygenation medium, the oxygenation constituent comprising a mixture of a first and second preparation wherein:
(i) the first preparation is a hemoglobin preparation comprising non-modified hemoglobin, HBOC, or a mixture thereof in an amount of from about 1% to about 40% by weight hemoglobin, the balance of the hemoglobin preparation comprising an aqueous hemoglobin diluent; and
(ii) the second preparation is a red blood cell preparation comprising hemoglobin in an amount of from about 1% to about 40% by weight hemoglobin, and the balance of the red blood cell preparation comprising an aqueous red blood cell diluent; and
wherein
the mixture is formed such that the oxygenation constituent comprises an amount of from about 10% to about 99% by weight hemoglobin of the hemoglobin preparation, and an amount equal to the balance by weight hemoglobin of the red blood cell preparation.

2. The oxygenation constituent according to claim 1, wherein the oxygenation constituent comprises from about 20% to about 90% by weight hemoglobin of the hemoglobin preparation.

3. The oxygenation constituent according to claim 1, wherein the oxygenation constituent comprises from about 30% to about 80% by weight hemoglobin of the hemoglobin preparation.

4. The oxygenation constituent according to claim 1, wherein the oxygenation constituent comprises from about 40% to about 70% by weight hemoglobin of the hemoglobin preparation.

5. The oxygenation constituent according to claim 1, wherein the oxygenation constituent comprises from about 50% to about 60% by weight hemoglobin of the hemoglobin preparation.

6. The oxygenation constituent according to claim 1, wherein the hemoglobin preparation comprises non-modified hemoglobin, HBOC, or a mixture thereof in an amount of from about 5% to about 35% by weight hemoglobin.

7. The oxygenation constituent according to claim 1, wherein the hemoglobin preparation comprises non-modified hemoglobin, HBOC, or a mixture thereof in an amount of from about 10% to about 30% by weight hemoglobin.

8. The oxygenation constituent according to claim 1, wherein the hemoglobin preparation comprises non-modified hemoglobin, HBOC, or a mixture thereof in an amount of from about 15% to about 25% by weight hemoglobin.

9. The oxygenation constituent according to claim 1, wherein the red blood cell preparation comprises hemoglobin in an amount of from about 5% to about 35% by weight hemoglobin.

10. The oxygenation constituent according to claim 1, wherein the red blood cell preparation comprises hemoglobin in an amount of from about 10% to about 30% by weight hemoglobin.

11. The oxygenation constituent according to claim 1, wherein the red blood cell preparation comprise hemoglobin in an amount of from about 15% to about 25% by weight hemoglobin.

12. The oxygenation constituent according to claim 1, wherein the oxygenation constituent comprises:
(i) a hemoglobin preparation comprising non-modified hemoglobin, HBOC or a mixture thereof in an amount of from about 5% to about 15% by weight hemoglobin, the balance of the hemoglobin preparation comprising an aqueous hemoglobin diluent; and
(iii) a red blood cell preparation comprising hemoglobin in an amount of from about 10% to about 30% by weight hemoglobin, and the balance of the red blood cell preparation comprising an aqueous red blood cell diluent; and
wherein
the mixture is formed such that the oxygenation constituent comprises an amount from about 40% to about 60% by weight hemoglobin of the hemoglobin preparation, and an amount equal to the balance by weight hemoglobin of the red blood cell preparation.

13. The oxygenation constituent according to claim 1, wherein the oxygenation constituent comprises:
(i) a hemoglobin preparation comprising non-modified hemoglobin, HBOC or a mixture thereof in an amount of about 10% by weight hemoglobin, the balance of the hemoglobin preparation comprising an aqueous hemoglobin diluent; and
(ii) a red blood cell preparation comprising hemoglobin in an amount of about 20% by weight hemoglobin, and the balance of the red blood cell preparation comprising an aqueous red blood cell diluent; and
wherein
the mixture is formed such that the oxygenation constituent comprises an amount of about 50% by weight hemoglobin of the hemoglobin preparation, and an amount equal the balance by weight hemoglobin of the red blood cell preparation.

14. The oxygenation constituent according to any one of claims 1 to 13, wherein the hemoglobin preparation comprises non-modified hemoglobin and is substantially free of HBOC.

15. The oxygenation constituent according to any one of claims 1 to 13, wherein the hemoglobin preparation comprises HBOC, and is substantially free of non-modified hemoglobin.

16. The oxygenation constituent according to any one of claims 1 to 13, wherein the hemoglobin preparation comprises a mixture of hemoglobin and HBOC.

17. The oxygenation constituent according to any one of claims 1 to 16, wherein the aqueous hemoglobin diluent is lactated Ringer's solution, or a modified form thereof.

18. The oxygenation constituent according to any one of claims 1 to 16, wherein the aqueous red blood cell diluent is lactated Ringer's solution, or a modified form thereof.

19. A finished oxygenation medium comprising:
(i) from about 1% by weight hemoglobin to about 39% by weight hemoglobin of an oxygenation constituent according to any one of claims 1 to 18; and
(ii) the balance of the medium comprising at least one other formulary ingredient selected from a diluent or an excipient, and optionally other ingredients suitable for formulating a finished oxygenation medium.

20. A finished oxygenation medium comprising:
(a) from about 1% by weight hemoglobin to about 39% by weight hemoglobin of an oxygenation constituent, the oxygenation constituent comprising a mixture of a first and second preparation wherein:
(i) the first preparation is a hemoglobin preparation comprising non-modified hemoglobin, HBOC, or a mixture thereof in an amount of from about 1% to about 40% by weight hemoglobin, the balance of the hemoglobin preparation comprising an aqueous hemoglobin diluent; and
(ii) the second preparation is a red blood cell preparation comprising hemoglobin in an amount of from about 1% to about 40% by weight hemoglobin, and the balance of the red blood cell preparation comprising an aqueous red blood cell diluent; and
wherein
the mixture is formed such that the oxygenation constituent comprises an amount of from about 10% to about 99% by weight hemoglobin of the hemoglobin preparation, and an amount equal to the balance by weight hemoglobin of the red blood cell preparation; and
(b) the balance of the medium comprising at least one other formulary ingredient selected from a diluent or an excipient, and optionally other ingredients suitable for formulating a finished oxygenation medium.

21. The finished oxygenation medium according to claim 20, wherein the finished oxygenation medium comprises from about 5% by weight hemoglobin to about 35% by weight hemoglobin of an oxygenation constituent.

22. The finished oxygenation medium according to claim 20, wherein the finished oxygenation medium comprises from about 10% by weight hemoglobin to about 30% by weight hemoglobin of an oxygenation constituent.

23. The finished oxygenation medium according to claim 20, wherein the finished oxygenation medium comprises from about 15% by weight hemoglobin to about 25% by weight hemoglobin of an oxygenation constituent, the oxygenation constituent comprising:
(i) a hemoglobin preparation comprising non-modified hemoglobin, HBOC or a mixture thereof in an amount of from about 5% to about 15% by weight hemoglobin, the balance of the hemoglobin preparation comprising an aqueous hemoglobin diluent; and
(ii) a red blood cell preparation comprising hemoglobin in an amount of from about 10% to about 30% by weight hemoglobin, and the balance of the red blood cell preparation comprising an aqueous red blood cell diluent; and
wherein
the mixture is formed such that the oxygenation constituent comprises an amount from about 40% to about 60% by weight hemoglobin of the hemoglobin preparation, and an amount equal to the balance by weight hemoglobin of the red blood cell preparation.

24. The finished oxygenation medium according to claim 20, wherein the finished oxygenation medium comprises about 20% by weight hemoglobin of an oxygenation constituent, the oxygenation constituent comprising:
(i) a hemoglobin preparation comprising non-modified hemoglobin, HBOC or a mixture thereof in an amount of about 10% by weight hemoglobin, the balance of the hemoglobin preparation comprising an aqueous hemoglobin diluent; and
(ii) a red blood cell preparation comprising hemoglobin in an amount of about 20% by weight hemoglobin, and the balance of the red blood cell preparation comprising an aqueous red blood cell diluent; and
wherein
the mixture is formed such that the oxygenation constituent comprises an amount of about 50% by weight hemoglobin of the hemoglobin preparation, and an amount equal the balance by weight hemoglobin of the red blood cell preparation.

25. A method of preparing a finished oxygenation medium for ex-vivo use, the method comprising:
(i) providing a hemoglobin preparation comprising non-modified hemoglobin, HBOC, or a mixture thereof in an amount of from about 1% to about 40% by weight hemoglobin, the balance of the hemoglobin preparation comprising an aqueous hemoglobin diluent;
(ii) providing a red blood cell preparation comprising hemoglobin in an amount of from about 1% to about 40% by weight hemoglobin, and the balance of the red blood cell preparation comprising an aqueous red blood cell diluent;
(iii) mixing the hemoglobin preparation and the red blood cell preparation to form an oxygenation constituent such that the oxygenation constituent comprises an amount of from about 10% to about 99% by weight hemoglobin of the hemoglobin preparation, and an amount equal to the balance by weight hemoglobin of the red blood cell preparation; and
(iv) formulating the oxygenation constituent with at least one formulary ingredient selected from an excipient, and a diluent, and optionally other ingredients suitable for formulating a finished oxygenation medium.

26. The method according to claim 25, wherein the finished oxygenation medium comprises at least about 1% by weight of hemoglobin of the oxygenation constituent.

27. The method according to claim 25, wherein the finished oxygenation medium comprises from about 1% to about 39% by weight of hemoglobin of the oxygenation constituent.

28. The method according to claim 25, wherein an amount of the hemoglobin preparation is mixed such that the oxygenation constituent comprises an amount of from about 20% to about 90% by weight hemoglobin of the hemoglobin preparation.

29. The method according to claim 25, wherein an amount of the hemoglobin preparation is mixed such that the oxygenation constituent comprises from about 30% to about 80% by weight hemoglobin of the hemoglobin preparation.

30. The method according to claim 25, wherein an amount of the hemoglobin preparation is mixed such that the oxygenation constituent comprises from about 40% to about 70% by weight hemoglobin of the hemoglobin preparation.

31. The method according to claim 25, wherein an amount of the hemoglobin preparation is mixed such that the oxygenation constituent comprises from about 50% to about 60% by weight hemoglobin of the hemoglobin preparation.

32. The method according to claim 25, wherein the hemoglobin preparation comprises non-modified hemoglobin, HBOC, or a mixture thereof in an amount of from about 5% to about 35% by weight hemoglobin.

33. The method according to claim 25, wherein the hemoglobin preparation comprises non-modified hemoglobin, HBOC, or a mixture thereof in an amount of from about 10% to about 30% by weight hemoglobin.

34. The method according to claim 25, wherein the hemoglobin preparation comprises non-modified hemoglobin, HBOC, or a mixture thereof in an amount of from about 15% to about 25% by weight hemoglobin.

35. The method according to claim 25, wherein the red blood cell preparation comprises hemoglobin in an amount of from about 5% to about 35% by weight hemoglobin.

36. The method according to claim 25, wherein the red blood cell preparation comprises hemoglobin in an amount of from about 10% to about 30% by weight hemoglobin.

37. The method according to claim 25, wherein the red blood cell preparation comprises hemoglobin in an amount of from about 15% to about 25% by weight hemoglobin.

38. The method according to claim 25, wherein, the oxygenation constituent comprises:
(i) a hemoglobin preparation comprising non-modified hemoglobin, HBOC or a mixture thereof in an amount of from about 5% to about 15% by weight hemoglobin, the balance of the hemoglobin preparation comprising an aqueous hemoglobin diluent; and
(ii) a red blood cell preparation comprising hemoglobin in an amount of from about 10% to about 30% by weight hemoglobin, and the balance of the red blood cell preparation comprising an aqueous red blood cell diluent; and
wherein
the mixture is formed such that the oxygenation constituent comprises an amount from about 40% to about 60% by weight hemoglobin of the hemoglobin preparation, and an amount equal to the balance by weight hemoglobin of the red blood cell preparation; and
(iii) the finished oxygenation medium comprises from about 15% by weight hemoglobin to about 25% by weight hemoglobin of the oxygenation constituent.

39. A use of an oxygenation constituent to formulate a finished oxygenation medium, the oxygenation constituent comprising:
a mixture of a first and second preparation wherein:
(i) the first preparation is a hemoglobin preparation comprising non-modified hemoglobin, HBOC, or a mixture thereof in an amount of from about 1% to about 40% by weight hemoglobin, the balance of the hemoglobin preparation comprising an aqueous hemoglobin diluent; and
(ii) the second preparation is a red blood cell preparation comprising hemoglobin in an amount of from about 1% to about 40% by weight hemoglobin, and the balance of the red blood cell preparation comprising an aqueous red blood cell diluent; and
wherein
the mixture is formed such that the oxygenation constituent comprises an amount of from about 10% to about 99% by weight hemoglobin of the hemoglobin preparation, and an amount equal to the balance by weight hemoglobin of the red blood cell preparation.

40. A use of a finished oxygenation medium for the ex-vivo preservation of a tissue or an organ, the finished oxygenation medium comprising:
(a) from about 1% by weight hemoglobin to about 39% by weight hemoglobin of an oxygenation constituent, the oxygenation constituent comprising a mixture of a first and second preparation wherein:
(i) the first preparation is a hemoglobin preparation comprising non-modified hemoglobin, HBOC, or a mixture thereof in an amount of from about 1% to about 40% by weight hemoglobin, the balance of the hemoglobin preparation comprising an aqueous hemoglobin diluent; and
(ii) the second preparation is a red blood cell preparation comprising hemoglobin in an amount of from about 1% to about 40% by weight hemoglobin, and the balance of the red blood cell preparation comprising an aqueous red blood cell diluent; and
wherein
the mixture is formed such that the oxygenation constituent comprises an amount of from about 10% to about 99% by weight hemoglobin of the hemoglobin preparation, and an amount equal to the balance by weight hemoglobin of the red blood cell preparation; and
(b) the balance of the medium comprising at least one other formulary ingredient selected from a diluent or an excipient, and optionally other ingredients suitable for formulating a finished oxygenation medium.

41. The use according to claim 40, wherein the ex-vivo preservation is static ex-vivo preservation.

42. The use according to claim 40, wherein the ex-vivo preservation is dynamic ex-vivo preservation.

43. The use according to claim 40, wherein the ex-vivo preservation is dynamic ex-vivo preservation involving the use of a machine perfusion system configured to comprise a filtering device to filter the finished oxygenation medium.

44. The use according to claim 40, wherein the ex-vivo preservation is dynamic ex-vivo preservation involving the use of a machine perfusion system configured to comprise a continuous or semi continuous supply assembly for the finished oxygenation medium, or an excipient or diluent, and a filtering device to continuously filter the finished oxygenation medium.

45. The use according to claim 43 or 44, wherein the filtering device is a tangential flow based filtering device.

46. The use according to any one of claims 40 to 45, wherein the organ is an organ for transplantation to a transplant recipient.

47. The use according to any one of claims 40 to 45, wherein the organ is a lung, a heart, a liver or a kidney.

48. The use according to any one of claims 40 to 45, wherein the organ is a human organ.

49. The use according to claim 40 wherein the tissue is for grafting to a graft recipient.

50. The use according to claim 40 wherein the tissue is for tissue engineering.

51. An organ preservation system comprising a circuitous perfusion fluid system operable to perfuse a perfusion fluid through an organ, the preservation system comprising a fluid reservoir to support an organ, the fluid reservoir in fluid connection with a continuous or semi-continuous supply assembly for supplying a perfusion fluid or an excipient to the fluid reservoir, the fluid reservoir further in fluid connection with a separation device permitting the filtration of the perfusion fluid upon removal of the perfusion fluid from the fluid reservoir to separate and remove spent constituents from the perfusion fluid, and a pump for circulating the perfusion fluid through the circuitous fluid system.

52. An organ preservation system according to claim 51 wherein the perfusion fluid is a hemoglobin based perfusion fluid.

53. An organ preservation system according to claim 51 wherein the perfusion fluid is a red-blood cell based perfusion fluid.

54. An organ preservation system according to claim 51 wherein the perfusion fluid is a finished oxygenation formulation according to any one of claims 19 to 24.

55. A composition, system and/or method comprising any combination of one or more of the features described above and/or claimed above.
